(19) United States
(12) Patent Application Publication
(10) Pub. No.: US 2014/0322364 A1
(43) Pub. Date: Oct. 30, 2014

(54) BODY TYPE SELF-ASSESSMENT AND TREATMENTS

(71) Applicant: David Yong Lee, Thousand Oaks, CA (US)

(72) Inventor: David Yong Lee, Thousand Oaks, CA (US)

(21) Appl. No.: 14/261,362

(22) Filed: Apr. 24, 2014

Related U.S. Application Data
(60) Provisional application No. 61/815,588, filed on Apr. 24, 2013.
(60) Provisional application No. 61/827,562, filed on May 25, 2013.

(51) Int. Cl. A61K 36/18; B65B 5/08; A61N 2/06

(52) U.S. Cl. CPC A61K 36/18; A61N 2/06; B65B 5/08
USPC 424/725; 600/15; 53/410

(57) ABSTRACT

Methods are provided for the self-assessment and treatment of medical conditions by an untrained layperson. Such methods may comprise determining the layperson's body type by self-assessment, selecting a mixture of herbs based upon the layperson's body type, and ingesting the mixture of herbs.

### RELATED APPLICATIONS

This application claims the benefit of: (i) U.S. Provisional Patent Application No. 61/815,588, filed on Apr. 24, 2013, and (ii) U.S. Provisional Patent Application No. 61/827,562, filed on May 25, 2013. The contents of both provisional applications are incorporated herein by reference in their entireties.

### FIELD OF THE APPLICATION

The present application relates to body type self-assessment and treatments based upon the results of the self-assessment, including herbal remedies and four body type magnet therapy.

### DESCRIPTION OF THE RELATED ART

Constitutional acupuncture has been popular since the 1960s. The most famous type of constitutional acupuncture is that of Dowon Kuon's, which was modeled by Dowon Kuon in the style of Saam. Saam created the four needle technique circa 1644-1742 CE. basing it on the idea of grandmother, mother, and child relationships with five elements put forth in the Classic of Difficulties written between 475 BCE-220 CE.

In 1894, Jema Lee published a text titled “Longevity and Life Preservation in Eastern Medicine” containing a systemized constitutional theory and herbal application. The book describes the four body types as: (a) Greater Yang, (b) Greater Yin, (c) Lesser Yang, and (d) Lesser Yin. In China, there are “yin”, “yang”, “hot” and “cold” body types as well as “wood”, “fire”, “earth”, “metal” and “water” body types. In India's Ayurveda, the body types are: (i) kapha, (ii) pitta, and (iii) vata. Through reinterpretation of Zhang Zhong Jing's cold-induced disorders, Jema Lee refined the Chinese herbal formulas to better fit the four constitutions. He also identified foods that are beneficial and harmful to the constitutions.

In 1965, Dowon Kuon converted Jema Lee's four constitutions to eight constitutions and applied the four needle technique which was an expansion from one tonification and one sedation methods to four tonification and four sedation methods. However, these methods are incongruent for at least the following reasons: (i) the method of diagnosing the constitutions is different between Dowon Kuon and Jema Lee; (ii) Dowon Kuon does not treat using herbal remedies and Jema Lee did not practice acupuncture on constitutional theory.

### BRIEF SUMMARY OF EMBODIMENTS OF THE APPLICATION

The present application describes a self-assessment used to determine body type. In some embodiments, the determination of body type can then be employed for the purpose of choosing herbal remedies and the packaging of herbal remedies by body type in multi-dose or unit-dose. In other embodiments, the determination of body type can be employed in four body type magnet therapy.

Some embodiments of this application involve the use of a questionnaire comprising a series of questions that a person answers and then scores to determine the person's body type. The questions may be in the form of a written questionnaire, a website questionnaire, a video questionnaire, audio questionnaire or other form of questionnaire. Using the self-assessment or with previous knowledge of body type, an untrained person is able to treat various conditions by using the novel pre-packaged herbal remedies labeled by body type, as set forth herein.

Other features and aspects of the application will become apparent from the following detailed description, taken in conjunction with the accompanying figures, which illustrate, by way of example, the features in accordance with embodiments of the application. The summary is not intended to limit the scope of the application, which is defined solely by the claims attached hereto.

### BRIEF DESCRIPTION OF THE FIGURES

The present application, in accordance with one or more various embodiments, is described in detail with reference to the following figures. The figures are provided for purposes of illustration only and merely depict typical or example embodiments of the application. These figures are provided to facilitate the reader's understanding of the disclosure and shall not be considered limiting of the breadth, scope, or applicability of the disclosure.

FIG. 1 is a diagram illustrating a method of self-assessment and treatment of medical conditions by an untrained layperson.

FIG. 2 is a diagram illustrating a method for packaging herbal remedies for use by an untrained layperson to self-treat a medical condition.

FIG. 3 is a diagram illustrating a method of self-assessment and treatment of a medical condition by an untrained layperson.

FIG. 4 is a diagram illustrating a method 400 for packaging magnets for use by an untrained layperson to self-treat a medical condition.

FIG. 5 is a diagram illustrating a first prescription for a Greater Yin+Lesser Yang body type. Specifically, the prescription comprises placing magnets at LI5 bionorth, SI5 bionorth, LI2 biosouth, UB66 biosouth.

FIG. 6 is a diagram illustrating a second prescription for a Greater Yin+Lesser Yang body type comprising placing magnets at LI2 bionorth, SI2 bionorth fire, LI11 biosouth, UB40 biosouth.

FIG. 7 is a diagram illustrating a first prescription for a Lesser Yang+Greater Yang body type comprising placing magnets at LI3 biosouth, GB41 biosouth, LI11 bionorth, ST36 bionorth.

FIG. 8 is a diagram illustrating a first prescription for a Lesser Yang+Greater Yang body type comprising placing magnets at LI1+biosouth, GB44 biosouth, LI3 bionorth, ST43 bionorth.

FIG. 9 is a diagram illustrating a first prescription for both Greater Yang and Lesser Yin body type and Greater Yin and Lesser Yin body type comprising placing magnets at SP2 biosouth, HT8 biosouth, SP9 bionorth, KD10 bionorth.

FIG. 10 is a diagram illustrating a first prescription for both Greater Yang and Lesser Yin body type Greater Yin and Lesser Yin body type comprising placing magnets at SP5 biosouth fire, HT4 biosouth, SP2 bionorth, KD2 bionorth.

These figures are not intended to be exhaustive or to limit the application to the precise form disclosed. It should be understood that embodiments of the application can be practiced with modification and alteration, and that the application be limited only by the claims and the equivalents thereof.

### DETAILED DESCRIPTION OF THE EMBODIMENTS OF THE APPLICATION

The present invention describes a self-assessment used to determine body type for the purpose of a person choosing herbal remedies and the packaging of herbal remedies by body type in multi-dose or unit-dose. More specifically the invention consists of a series of questions that a person answers and then scores to determine the person's body type.

Throughout the world, the terms “body type”, “constitution”, and “body constitution” are used substantially interchangeably. For example, “body type” is used in India's Ayurveda whereas “constitution” is used in Korea's Four Constitutional Medicine. Although the term “constitution” is wider and more encompassing, both “constitution” and “body type” are interchangeably used by the lay public and complementary practitioners. As used herein, the term “body type” means a human body shape and physique type, while the term “constitution” means an aggregate of a person's physical, physiological and psychological characteristics. The term “body type” is used interchangeably with “constitution”.

Body type determination is important in the choice of herbal remedies. Properly identifying the individual's body type minimizes the potential side effects and produces results that are more effective. No herbal remedy or any product is effective for every person and there is variation in the individual response. Identifying the best fitting herbal ingredients and therefore making an herbal blend according to the body type (in addition to the symptomatic and biochemical indications) significantly increase the resultant efficacy. The result is a reduction of symptoms and increased health benefit.

Some embodiments of the invention involve a method for an untrained person to determine their body type in the context of traditional oriental herbal remedy. By way of example, this method may be in the form of a written questionnaire as set forth herein. In operation, a person self-administers and then scores the questionnaire to determine body type. Alternatively, the questionnaire may comprise an audio version of the questionnaire. According to further embodiments, the self-assessment may be in the form of pictures representing a person's body or body parts that a person selects as answers. The person then scores the picture-based self-assessment based on their selections.

Additional embodiments of the invention involve methods of packaging herbal remedies labeled by body type. Such methods may entail multi-dose packaging to be measured by a consumer, as well as unit-dose packaging for use by a consumer that does not require measuring.

The following are examples of a written questionnaire question including a questionnaire example for each body type:

Greater Yang questionnaire example: I am allergic to nuts. I have one or more symptoms of scratchy throat, throat swelling, itchy skin, sneezing, and post-nasal drip if when I continue to consume them.

Greater Yin questionnaire example: I have allergies to shellfish (shrimp, clam, mussel). I have one or more symptoms of scratchy throat, swelling in the mouth, and itchy skin.

Lesser Yang questionnaire example: I am diagnosed with ADHD, Attention Deficit Hyperactive Disorder. Or I have a mild form of it.

Lesser Yin questionnaire example: I have gluten intolerance. Wheat products make me bloated or cause irritable bowel symptoms. Other symptoms may be fatigue, head foggy sensation, and difficulty focusing.

In some embodiments, a questionnaire can be used to identify the four body types. It may involve a scoring system where the two categories of questions identify two aspects of the body type. The following are example questions to determine Greater Yang (a) vs. Greater Yin (b) body type:
1a. I find steaks to be heavy and take a long time to digest. My bowels are often affected. I do not like the sensation from a high protein diet, which makes me feel heavy, tired, and bloated.
b. I do not find steaks to be heavy. I digest them quickly and my bowels do not change. I benefit from a high protein diet resulting in weight loss, and increase in energy, focus, and clarity.
2a. I find fried foods to be heavy and take a long time to digest. My bowels are often easily affected.
b. I do not find greasy or fried foods to be heavy. I digest them quickly and my bowels do not change.
3a. I get motion sickness easily, such as on boats or airplanes. I cannot read a book in the car as a passenger for more than five minutes.
b. I do not get motion sickness easily, such as on boats or airplanes. I can read as a passenger in a car for more than 15 minutes.
4a. I am lactose intolerant or allergic to milk products. I experience increased nasal discharge, bloating, and or a change in bowel function after drinking a glass of milk.
b. I am not allergic to milk products. I do not feel bloated or have a change in bowel movement. I can drink two glasses of milk every day without symptoms.
5a. My sense of hearing is keener than my sense of smell.
b. My sense of smell is keener than my sense of hearing.
6a. A strong mint taste, such as mint flavored gum, when it is in the mouth for more than ten minutes, makes me feel light headed or dizzy.
b. A strong mint taste, such as mint flavored gum, when it is in the mouth for more than ten minutes, does not make me feel light headed or dizzy.
7a. I do not like perfume or strong odor. They sometimes give me a headache or make me dizzy.
b. I am not bothered by perfume or strong odors.
8a. Regardless of the quality of my voice, my voice is middle to high pitched.
b. Regardless of the quality of my voice, my voice is middle to low pitched.
9a. I would rather be warm than cold.
b. I would rather be cold than warm.
10a. I have difficulty ignoring a nagging pain, which affects my activity.
b. When distracted, I often forget a nagging pain.
11a. I feel uncomfortable wearing a turtleneck or a scarf around my neck even in cooler weather. I use it as a fashion statement only.
b. I feel comfortable wearing a scarf or turtleneck around my neck in cooler weather. I often wear them for fashion statement.
12a. I get bothered if I wear a hat all day.
b. Wearing a hat makes me feel comfortable and cozy. I can wear a hat all day long.
13a. I do not like to talk or to sing for a long time because my voice tends to get hoarse easily.
b. I can talk or sing for a long time. My voice does not easily get hoarse and my throat is not easily affected.
14a. I do not like strenuous physical activities. I have to put in extra effort.
b. I like physical activities that require strength or stamina.
15a. My shoes tend to last a long time and stay in pristine shape.
b. My shoes tend to break down easily, especially on the sides.
16a. I cannot stay awake all night to finish a project.
b. I can stay up all night to finish a project.
17a. I prefer to sit and do not like to stand for a long time.
b. I can stand for a long time and do not need to seat.
18a. I am a night person. I tend to stay up late and get up late.
b. I am a morning person. I go to bed early and get up early.
19a. I tend to act first and plan as I go along.
b. I tend to think things through thoroughly and plan before acting.
20a. I am uneasy at heights and it takes time for me to adjust.
b. I have no problem with heights and can work on high places with relative ease.
21a. I am allergic to nuts. I have one or more symptoms of scratchy throat, itchy skin, sneezing, throat swelling, or post-nasal drip if when I consume them.
b. I have allergies to shellfish (shrimp, clam, mussel). I have one or more symptoms of scratchy throat, swelling in the mouth, and itchy skin.
22b. I have/had gall stones.

The following are example questions to determine Lesser Yang (c) vs. Lesser Yin (d) body type:
1c. I find myself almost never skipping meals. If I skip a meal, I get easily agitated and irritated.
d. I often find myself forgetting to eat meals. I skip meals regularly and do not feel strong hunger on an empty stomach.
2c. I cannot eat slowly. In a group of 10 people, I am one of the first three to finish a meal.
d. In a group of 10 people, I am one of the last three to finish a meal.
3c. I am not picky with foods. I tend to try new dishes and flavors.
d. I am finicky with foods. There are many food items or dishes I avoid.
4c. I like mint flavored gum or candy more than cinnamon.
d. I like cinnamon flavored gum or candy more than mint.
5c. Generally, I find myself rushing to accomplish at the last moment, instead of step-by-step increments.
d. Generally, I do my tasks on step-by-step increments.
6c. It is a challenge to keep a regular diary.
d. I do keep or can keep a regular diary.
7c. I prefer to live without a daily schedule on my phone or organizer.
d. I cannot imagine living a smooth life without a daily schedule on my phone or organizer.
8c. It is cumbersome to keep track of detailed information, and I am not detail oriented.
d. I am good at paying attention to minute details; I do not mind it being a requirement of my career.
9c. My room is often disorganized but I can easily find what I need.
d. My room is usually organized and I cannot think clearly if it is messy.
10c. I am usually the first to try something new, rather than using someone else to be a guinea pig.
d. I would rather have somebody else try something new before deciding. I am usually not the first to try.
11c. I have ADHD, Attention Deficit Hyperactive Disorder or a mild form of it.
d. I have obsessive compulsive disorder or a mild form of it.
12c. When I was a baby, I generally slept through the night.
d. When I was a baby, I woke up often during the night.
13c. I can sleep anywhere and sleep deeply.
d. I am finicky about where I sleep and am a light sleeper.
14c. When I am extremely tired, I fall asleep very easily.
d. When I am extremely tired, I find it difficult to fall asleep.
15c. Caffeine generally does not affect my sleep.
d. Caffeine will cause insomnia if I drink it after 3:00 pm.
16c. Even in cold weather, I cannot sleep with blanket over my face. I feel confined.
d. In cold weather, I often like to sleep with blanket over my face.
17c. My hands and feet are usually warm. When they get cold, they quickly warm back up.
d. My hands and feet get cold easily and tend to stay cold.
18c. Drinking ice water does not cause cramps or bloating. I can drink it all day.
d. Drinking ice water causes cramping or bloating. It occurs immediately or after a few days of consistently drinking it.
19c. I had a soy allergy as an infant or as an adult which might have ranged from skin itching or to anaphylactic shock.
20d. I have gluten intolerance. Wheat products make me bloated or cause irritable bowel symptoms.
21d. I always had thick set of hair. I have many follicles in my scalp which contains two or three hair roots. My hair density is in the top twenty percentile of the population.
22d. I have a history of being anemic (mild to severe) or am prone to anemia.
23d. I am or was diagnosed with gastroparesis, which is a slow emptying of food from stomach into small intestine.

After completing the questionnaire, the numbers are tallied by adding up the number of check marks for each body type. The two highest counts indicate the person's body type combination. If the difference between the totals of the two body types is four or less, the person may want to consult with a constitutional practitioner to confirm the diagnosis. The resulting body type combination will be one of these four combinations: (i) Greater Yang+Lesser Yang, (ii) Greater Yang+Lesser Yin, (iii) Greater Yin+Lesser Yang, and (iv) Greater Yin+Lesser Yin.

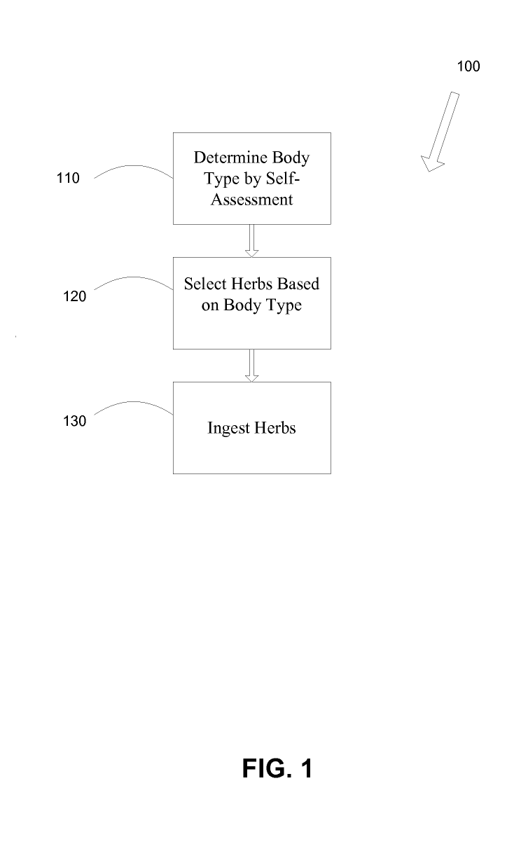

Referring to FIG. 1, an embodiment of the invention involves a method 100 of self-assessment and treatment of medical conditions by an untrained layperson. Specifically, step 110 comprises determining the layperson's body type by self-assessment. Step 120 comprises selecting a mixture of herbs based upon the layperson's body type, while step 130 comprises ingesting the mixture of herbs.

In some embodiments, the step of selecting the mixture of herbs based upon the layperson's body type comprises selecting a package of herbs containing a label that identifies the layperson's body type. The self-assessment can be in the form of a written questionnaire, a choice of pictures presented by illustration, video or computer program. Alternatively, it may be in the form of audio questions and descriptions.

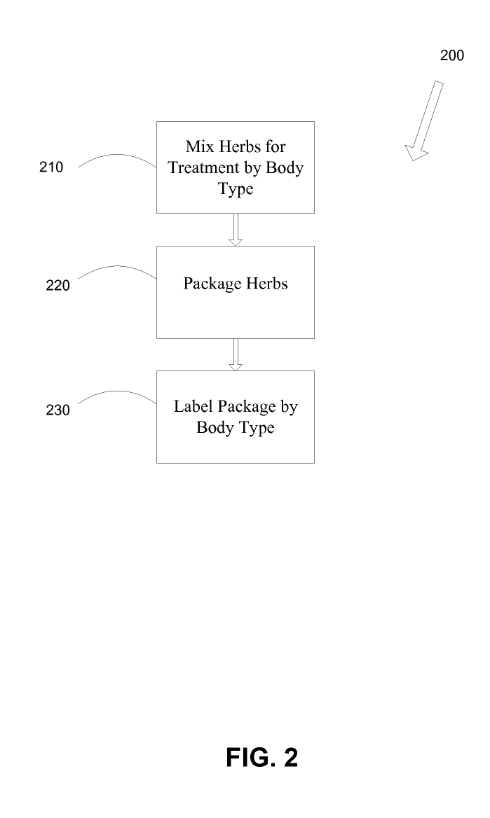

Referring to FIG. 2, another embodiment of the invention involves a method 200 for packaging herbal remedies for use by an untrained layperson to self-treat a medical condition. Step 210 of the method comprises mixing herbs in a proprietary blend for the treatment of the medical condition by body type. Step 220 entails packaging the herbs into a package. Step 230 comprises labeling the package to be for a particular body type.

In some embodiments, packaging the herbs comprises multi-dose packaging of herbal remedies. In other embodiments, packaging the herbs can comprise unit-dose packaging of herbal remedies. The body type is selected from the group consisting of: Greater Yang, Greater Yin, Lesser Yang, and Lesser Yin.

An additional embodiment of the invention involves a method for preparing herbal formulas including labeling for a particular body type. Specifically, the method may comprise providing herbs that meet all the basic requirements for government regulations to be sold online and store shelves. The next step involves mixing the herbs in a proprietary blend. Such a proprietary blend can be formulated by David Lee or be based on an exact formula as written in the Chinese herbal textbooks. The herbs can be in a form of tablet, capsule, gel, or tincture which is taken by mouth. In addition, the herbs can be packaged in a typical style of supplement and herbal products seen on outside shelves of pharmacies.

The next step of the method entails placing the herbs in a labeled container. The label can be used to identify one of the four body types, i.e., (a) Greater Yang, (b) Greater Yin, (c) Lesser Yang, and (d) Lesser Yin. In other words, a container labeled “Lesser Yang” contains herbs that are specifically chosen for a person having a Lesser Yang body type. The label may also identify the herbs as having passed the strict standards of quality of a natural product. In some embodiments, container may include packaging with instructions and/or a website address providing the person with access to the body type questionnaire and information on self-diagnosis. In embodiments featuring multi-dose packaging, the instructions can include information for measuring a proper dose. Embodiments featuring unit-dose packaging do not require measuring.

In general, the Lesser Yin herbs are shared by both: (i) Greater Yang and Lesser Yin body type, and (ii) Greater Yin and Lesser Yin body type. Greater Yang and Lesser Yang body type has its own herbs, and Greater Yin and Lesser Yang body type has its own herbs. The herbs can help to relieve ailments such as indigestion, headache, low back pain, menopausal symptoms and sinusitis.

Further embodiments of the invention are directed toward methods for the placement of magnets on acupuncture points according to constitution or body type, and the preparation and packaging of magnets by constitution. A layperson can determine their constitution as set forth hereinabove using a body type questionnaire, whereby a series of questions that the person answers are then scored to determine the person's constitution. The invention also describes the preparing and packaging of magnets by constitution for use by a layperson. The layperson may choose one or several given acupuncture prescriptions.

Constitution is important in the choice of magnet therapies. Properly identifying the individual's constitution minimizes the potential side effects and produces more effective results. No magnet therapy or any product is effective for every person, and there is significant variation in the individual response. Efficacy is increased by identifying the best fitting acupuncture prescription according to the constitution and the symptomatic and biochemical indications. The result is an increase in the reduction of symptoms and health benefit.

The four constitutions used in constitutional acupuncture are (a) Greater Yang, (b) Greater Yin, (c) Lesser Yang, and (d) Lesser Yin. Additionally, combinations of these constitutions can be employed, including: (e) Greater Yang+Lesser Yang, (f) Greater Yang+Lesser Yin, (g) Greater Yin+Lesser Yang, and (h) Greater Yin+Lesser Yin. Other cultures and therapies may have different names for these constitutions and this invention is expressly not limited to the Chinese names. As set forth above, the questions may be in the form of a written questionnaire, a website questionnaire, a video questionnaire, audio questionnaire or other form of questionnaire. An untrained person is able to treat various conditions using this invention's pre-packaged magnets labeled by constitution provided that the person initially determines their constitution by using a suitable questionnaire such as set forth herein. Alternatively, the person might have previous knowledge of their constitution, or has been provided with prescribed choices.

According to one embodiment of the invention, magnets are placed on one or more of the 50 acupuncture points as listed the below ten groups:
1. Liver 1 dadun, Liver 2 xingjian, Liver 3 taichong, Liver 4 zhongfeng, Liver 8 ququan
2. Heart 9 shaochong, Heart 8 shaofu, Heart 7 shenmen, Heart lingdao, Heart 3 shaohai
3. Spleen 1 yinbai, Spleen 2 dadu, Spleen 3 taibai, Spleen 5 shangqiu, Spleen 9 yinlingquan
4. Lung 11 shaoshang, Lung 10 yuji, Lung 9 taiyuan, Lung 8 jingqu, Lung 5 chize
5. Kidney 1 yongquan, Kidney 2 ranggu, Kidney 3 taixi, Kidney 7 fuliu, Kidney 10 yingu
6. Gall Bladder 44 zhuqiaoyin, Gall Bladder 43 xiaxi, Gall Bladder 41 zulinqi, Gall Bladder 38 yangfu, Gall Bladder 34 yanglingquan
7. Small Intestine 1 shaoze, Small Intestine 2 qiangu, Small Intestine 3 houxi, Small Intestine 5 yanggu, Small Intestine 8 xiaohai
8. Stomach 45 lidui, Stomach 44 neiting, Stomach 43 xiangu, Stomach 41 jiexi, Stomach 36 zusanli
9. Large Intestine 1 shangyang, Large Intestine 2 erjian, Large Intestine 3 sanjian, Large Intestine 5 yangxi, LH 1 quchi
10. Urinary Bladder 67 zhiyin, Urinary Bladder 66 zutonggu, Urinary Bladder 65 shugu, Urinary Bladder 60 kunlun, Urinary Bladder 40 weizhong

In operation, magnets are differentiated by the polarity. In particular, the biosouth side of the magnet facing the acupuncture point has a tonifying effect, whereas the bionorth side has a sedating effect when facing the acupuncture point. According to various embodiments, all magnet sizes and gauss strengths can be used.

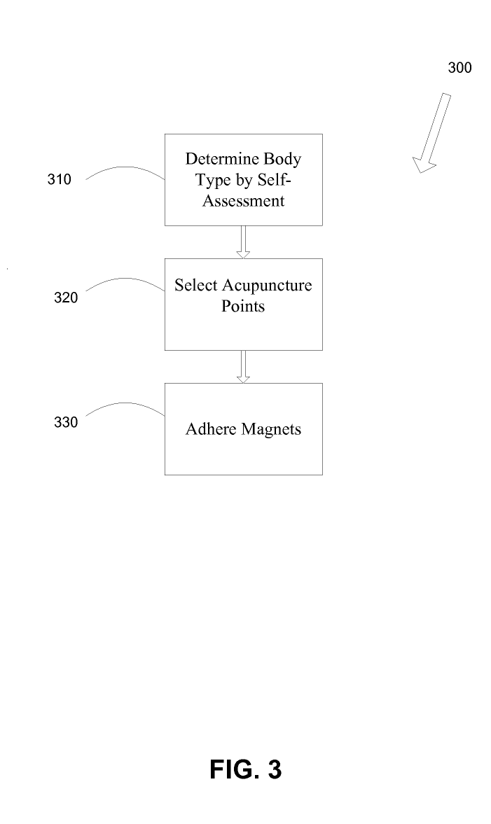

Referring to FIG. 3, a method 300 of self-assessment and treatment of a medical condition by an untrained layperson will now be described. In particular, step 310 comprises determining the layperson's body type by self-assessment. Step 320 comprises selecting a plurality of acupuncture points on the layperson on which to adhere magnets to self-treat the medical condition. Step 330 comprises adhering the magnets on the acupuncture points.

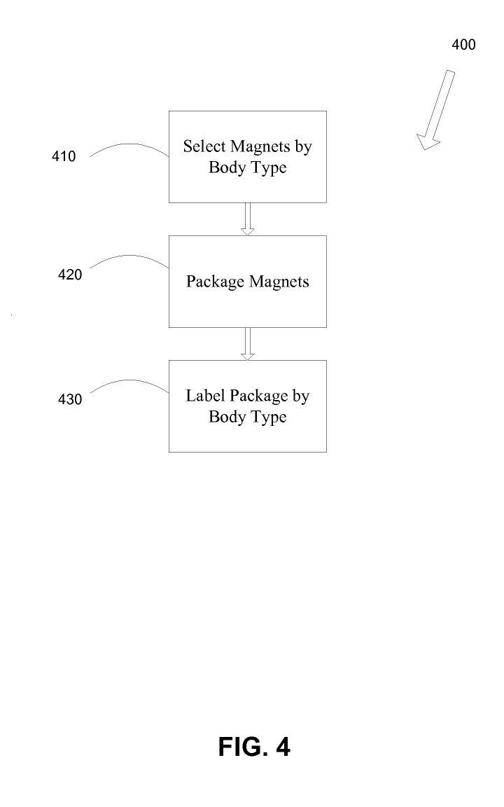

Referring to FIG. 4, a method 400 for packaging magnets for use by an untrained layperson to self-treat a medical condition will now be described. Specifically, step 410 entails selecting a plurality of magnets for the treatment of the medical condition by body type. Step 420 comprises packaging the magnets into a package, and step 430 comprises labeling the package to be for a particular body type.

According to a further embodiment of the invention, a method for packaging magnets by constitution for use by an untrained person to self-treat medical conditions will now be described. The initial step comprises providing a predetermined series of magnets. In some embodiments, the magnets can be disc-shaped magnets having a width of about 6 mm and a thickness of about 1.5 mm to about 2.0 mm. Additionally, in some embodiments, the faces of the magnets are color coded to identify the bionorth and biosouth polarities. By way of example, the magnets can be common earth magnets, or neodymium materials, magnetized prior to packaging.

The next step of the method involves providing packaging for the magnets, including instructions for self-diagnosis. In some embodiments, the packaging includes instructions and/or a website address providing the person with access to the body type questionnaire and information on self-diagnosis. The subsequent step entails labeling the magnets and/or packaging. The label can be used to identify one of the four body types, i.e., (a) Greater Yang, (b) Greater Yin, (c) Lesser Yang, and (d) Lesser Yin. In other words, a package labeled “Lesser Yang” contains magnets that are specifically chosen for a person having a Lesser Yang body type. The label may also identify the strict standards of quality such as containing non-allergenic adhesive and metal, recycled material, and recyclable material.

According to some embodiments, the content of a packages can include one or more of the following items: (i) packaging material, (ii) label, (iii) instruction sheet, (iv) magnets, (v) magnet holder/retainer/dispenser, (vi) adhesives, (vii) adhesive holder/retainer/dispenser.

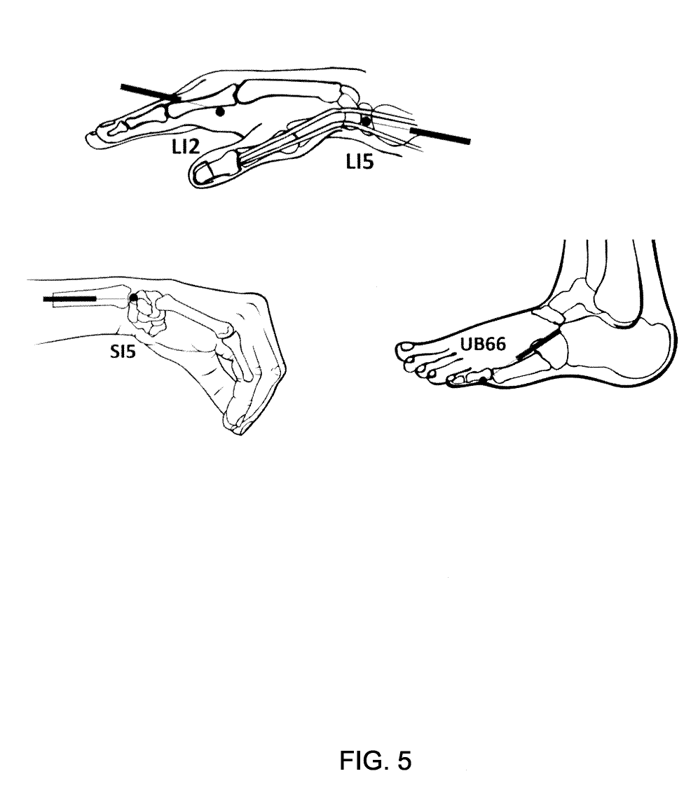

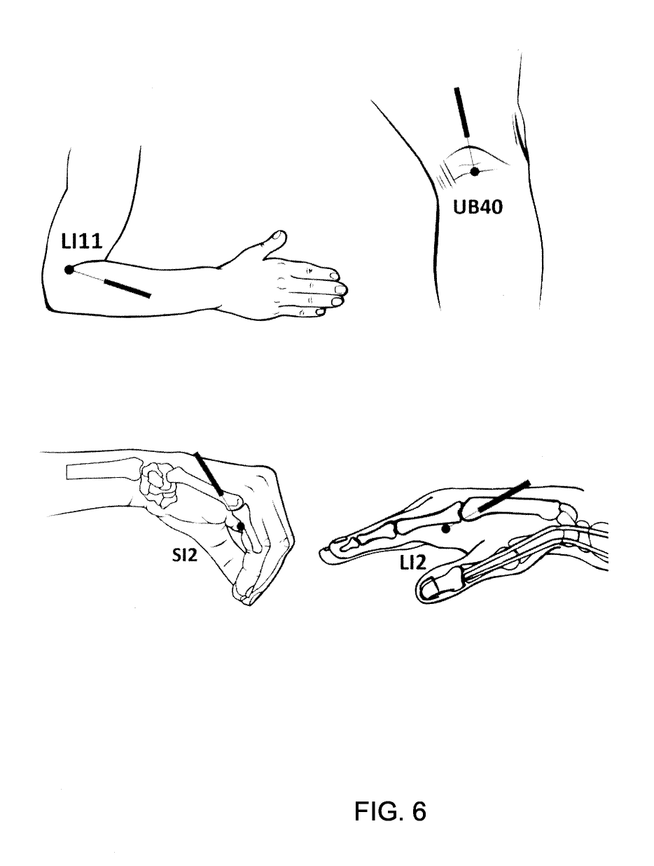

FIG. 5 is a diagram illustrating a first prescription for a Greater Yin+Lesser Yang body type. Specifically, the prescription comprises placing magnets at LI5 bionorth, SI5 bionorth, LI2 biosouth, UB66 biosouth. FIG. 6 is a diagram illustrating a second prescription for a Greater Yin+Lesser Yang body type comprising placing magnets at LI2 bionorth, SI2 bionorth fire, LI11 biosouth, UB40 biosouth.

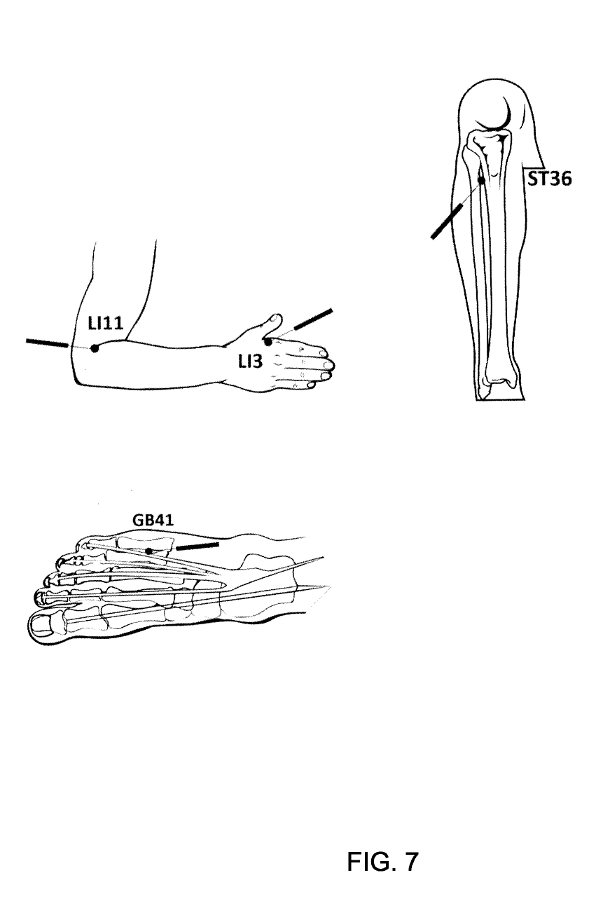

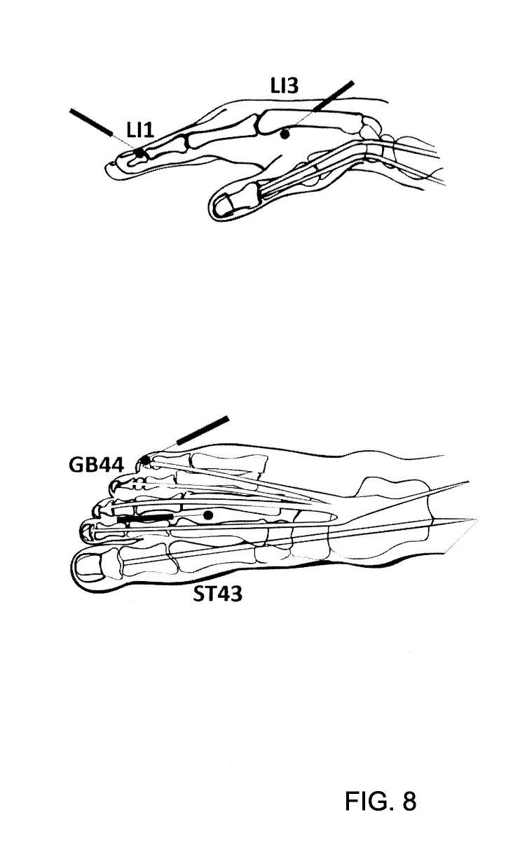

FIG. 7 is a diagram illustrating a first prescription for a Lesser Yang+Greater Yang body type comprising placing magnets at LI3 biosouth, GB41 biosouth, LI11 bionorth, ST36 bionorth. FIG. 8 is a diagram illustrating a first prescription for a Lesser Yang+Greater Yang body type comprising placing magnets at LI1+biosouth, GB44 biosouth, LI3 bionorth, ST43 bionorth.

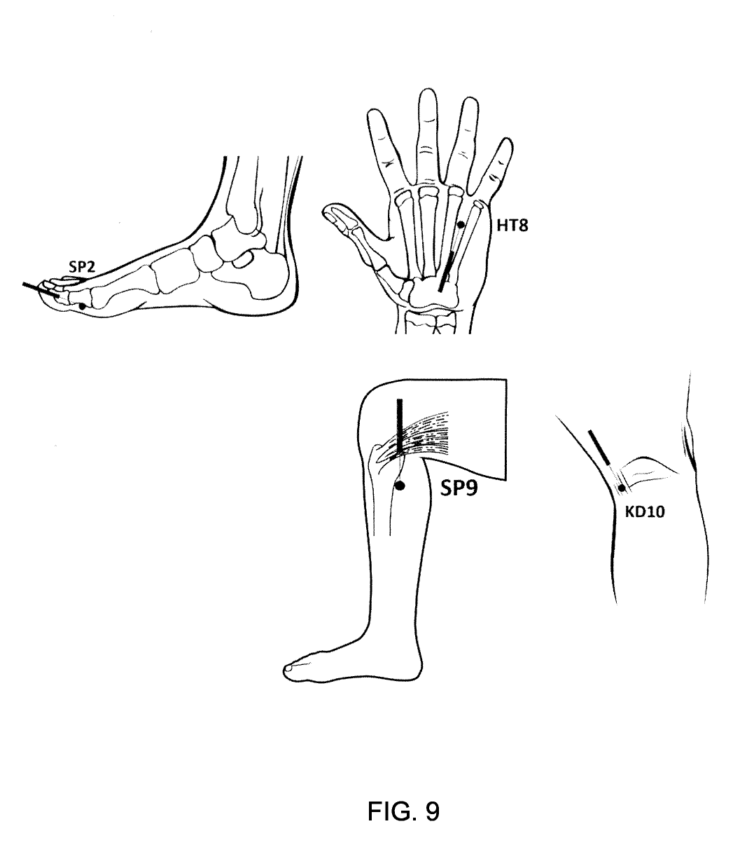

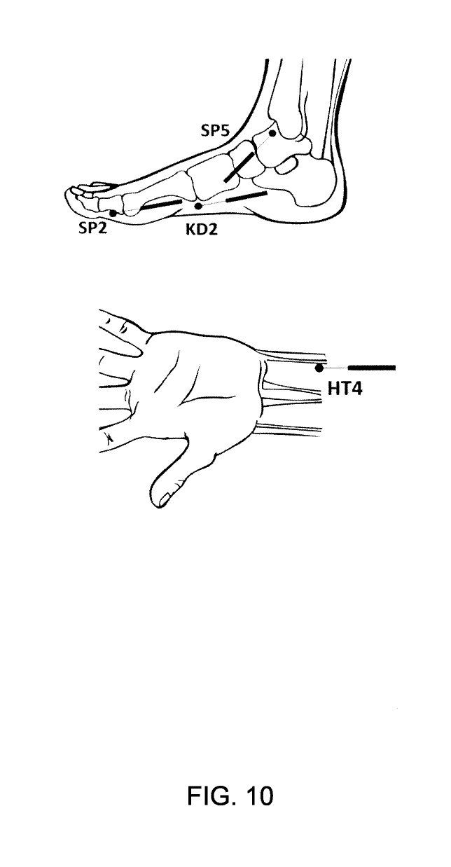

FIG. 9 is a diagram illustrating a first prescription for both Greater Yang and Lesser Yin body type and Greater Yin and Lesser Yin body type comprising placing magnets at SP2 biosouth, HT8 biosouth, SP9 bionorth, KD10 bionorth. FIG. 10 is a diagram illustrating a first prescription for both Greater Yang and Lesser Yin body type Greater Yin and Lesser Yin body type comprising placing magnets at SP5 biosouth fire, HT4 biosouth, SP2 bionorth, KD2 bionorth.

A method for preparing and packaging magnets by constitution for use by an untrained person to self-treat medical conditions will now be described. According to one embodiment of the invention, magnets are placed on one of the following six acupuncture point prescriptions:

### 1. LI5, SI5, LI2, UB66

### 2. LI2, SI2, LI11, UB40

### 3. LI3, GB41, LI11, ST36

### 4. LI1, GB44, LI3, ST43

### 5. SP2, HT8, SP9, KD10

### 6. SP5, HT4, SP2, KD2

An additional embodiment involves a method of self-assessment for use by an untrained person to determine body type for the purpose of choosing acupuncture points on which to adhere magnets to self-treat medical conditions. The packaging can be selected according to a person's constitution, and the self-assessment can be in the form of a written questionnaire. Alternatively, the self-assessment can be in the form of a choice of pictures presented by illustration, video or computer program. As a further alternative, the self-assessment can be in the form of audio questions and descriptions.

While various embodiments of the present application have been described above, it should be understood that they have been presented by way of example only, and not of limitation. Likewise, the various diagrams may depict an example architectural or other configuration for the disclosure, which is done to aid in understanding the features and functionality that can be included in the disclosure. The application is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present application. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the application is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A method of self-assessment and treatment of medical conditions by an untrained layperson, comprising:
determining the layperson's body type by self-assessment;
selecting a mixture of herbs based upon the layperson's body type; and
ingesting the mixture of herbs.

2. The method of claim 1, wherein selecting the mixture of herbs based upon the layperson's body type comprises selecting a package of herbs containing a label that identifies the layperson's body type.

3. The method of claim 1, wherein the self-assessment is in the form of a written questionnaire.

4. The method of claim 1, wherein the self-assessment is in the form of a choice of pictures presented by illustration, video or computer program.

5. The method of claim 1, wherein the self-assessment is in the form of audio questions and descriptions.

6. A method for packaging herbal remedies for use by an untrained layperson to self-treat a medical condition, comprising:
mixing herbs in a proprietary blend for the treatment of the medical condition by body type;
packaging the herbs into a package; and
labeling the package to be for a particular body type.

7. The method of claim 6, wherein packaging the herbs comprises multi-dose packaging of herbal remedies.

8. The method of claim 6, wherein packaging the herbs comprises unit-dose packaging of herbal remedies.

9. The method of claim 6, wherein the body type is selected from the group consisting of: Greater Yang, Greater Yin, Lesser Yang, and Lesser Yin.

10. The method of claim 6, wherein the package includes instructions or a website address providing the person with access to a body type questionnaire and information on self-diagnosis.

11. A method of self-assessment and treatment of a medical condition by an untrained layperson, comprising:
determining the layperson's body type by self-assessment;
selecting a plurality of acupuncture points on the layperson on which to adhere magnets to self-treat the medical condition.

12. The method according to claim 11, wherein the self-assessment is in the form of a written questionnaire.

13. The method of claim 1, wherein the self-assessment is in the form of a choice of pictures presented by illustration, video or computer program.

14. The method of claim 1, wherein the self-assessment is in the form of audio questions and descriptions.

15. A method for packaging magnets for use by an untrained layperson to self-treat a medical condition, comprising:
selecting a plurality of magnets for the treatment of the medical condition by body type;
packaging the magnets into a package; and
labeling the package to be for a particular body type.

16. The method of claim 15, wherein the body type is selected from the group consisting of: Greater Yang, Greater Yin, Lesser Yang, and Lesser Yin.

17. The method of claim 15, wherein the package includes instructions or a website address providing the person with access to a body type questionnaire and information on self-diagnosis.
